(19) United States
(12) Patent Application Publication
(10) Pub. No.: US 2026/0325352 A1
(43) Pub. Date: Oct. 22, 2026

(54) ALIGNING A CAPSULOTOMY PATTERN WITH AN EYE

(71) Applicant: ALCON INC., Fribourg (CH)

(72) Inventors: Parisa RABBANI, Aliso Viejo, CA (US); Lingfeng YU, Rancho Santa Margarita, CA (US)

(21) Appl. No.: 19/642,019

(22) Filed: Apr. 8, 2026

Related U.S. Application Data
(60) Provisional application No. 63/791,881, filed on Apr. 21, 2025.

(51) Int. Cl. A61F 9/008; A61B 3/08; A61B 3/14

(52) U.S. Cl. CPC A61F 9/008; A61B 3/085; A61B 3/14; A61F 2009/00889

(57) ABSTRACT

In at least one embodiment, an ophthalmic system comprises a laser system, an imaging system, and a computer system. The laser system provides a laser beam to perform a capsulotomy procedure on an eye. The laser system has an optical axis. The imaging system generates an image of the eye. The computer system determines a tilt-offset of the eye from the image of the eye, where the tilt-offset of the eye is relative to a z-axis corresponding to the optical axis. The computer system adjusts a capsulotomy pattern to compensate for the tilt-offset of the eye and instructs the laser system to provide the laser beam according to the capsulotomy pattern adjusted to compensate for the tilt-offset of the eye.

### FIELD

Embodiments of the present disclosure relate to aligning a capsulotomy pattern with an eye to perform a capsulotomy procedure.

### BACKGROUND

In cataract surgery, an ophthalmic system may use a laser system to perform a capsulotomy procedure. In an example, the laser system makes an incision in the capsule of the crystalline lens of the eye according to a capsulotomy pattern to create an opening in the capsule. The crystalline lens is removed from the capsule, and an intraocular lens (IOL) implant is placed into the capsule.

### SUMMARY

In at least one embodiment, an ophthalmic system comprises a laser system, an imaging system, and a computer system. The laser system provides a laser beam to perform a capsulotomy procedure on an eye. The laser system has an optical axis. The imaging system generates an image of the eye. The computer system determines a tilt-offset of the eye from the image of the eye, where the tilt-offset of the eye is relative to a z-axis corresponding to the optical axis. The computer system adjusts a capsulotomy pattern to compensate for the tilt-offset of the eye and instructs the laser system to provide the laser beam according to the capsulotomy pattern adjusted to compensate for the tilt-offset of the eye.

One or more embodiments may include none, one, two or more, or all of the following:

The computer system determines the tilt-offset of the eye from the image of the eye by identifying an eye structure in the image of the eye and determining the tilt-offset of the eye according to the eye structure and the z-axis. The eye structure may be a lens, an iris, and/or a cornea of the eye.

The computer system determines the tilt-offset of the eye from the image of the eye by identifying an eye axis from the image of the eye and determining the tilt-offset of the eye according to the eye axis and the z-axis. The computer system may identify the eye axis from the image by identifying a vertex of a cornea in the image of the eye, identifying a nodal point of the cornea in the image of the eye, and determining a visual axis according to the vertex of the cornea and the nodal point of the cornea. The computer system may identify the eye axis from the image by identifying a fovea of a retina in the image of the eye, identifying a nodal point of a cornea in the image of the eye, and determining a visual axis according to the fovea of the retina and the nodal point of the cornea. The computer system may identify the eye axis from the image by identifying a corneal surface of a cornea in the image of the eye, identifying a lenticular surface of a lens in the image of the eye, and determining an optical axis according to the corneal surface of the cornea and the lenticular surface of the lens. The computer system may identify the eye axis from the image by identifying a pupil center of a pupil in the image of the eye, identifying a nodal point of a cornea in the image of the eye, and determining a pupillary axis according to the pupil center of the pupil and the nodal point of the cornea. The computer system may identify the eye axis from the image by identifying a first preliminary eye axis in the image of the eye, identifying a second preliminary eye axis in the image of the eye, and determining the eye axis according to the first preliminary eye axis and the second preliminary eye axis. The computer system may identify the eye axis from the image by receiving a selection of the eye axis and using the selection as the eye axis to identify in the image of the eye.

One or more embodiments may include none, one, two or more, or all of the following:

The computer system determines an xy-offset of the eye from the image of the eye, where the xy-offset is relative to a central xy-point of an xy-plane defined relative to the z-axis. The computer system then adjusts the capsulotomy pattern to compensate for the xy-offset of the eye. The computer system may determine the xy-offset of the eye from the image of the eye by identifying an eye center from the image of the eye and determining a difference between the eye center and the central xy-point.

The computer system identifies an eye center from the image of the eye and determines an xy-offset of the eye according to the eye center and a central xy-point of an xy-plane defined relative to the z-axis. The computer system may identify the eye center from the image of the eye by identifying a pupil in the image of the eye and determining a pupil center of the pupil as the eye center. The computer system may identify the eye center from the image of the eye by identifying a lens capsule in the image of the eye and determining a lens capsule center of the lens capsule as the eye center. The computer system may identify the eye center from the image of the eye by identifying corneal angles in the image of the eye (where a corneal angle is formed from a posterior surface of a cornea of the eye and an anterior surface of an iris of the eye) and determining an angle center according to the corneal angles as the eye center. The computer system may identify the eye center from the image of the eye by determining an eye axis of the image of the eye and determining the eye center according to the eye axis. The computer system may identify the eye center from the image of the eye by receiving a selection of the eye center and identifying the selection as the eye center to identify in the image of the eye. The computer system may identify the eye center from the image of the eye by identifying a first preliminary eye center in the image of the eye, identifying a second preliminary eye center in the image of the eye, and determining the eye center according to the first preliminary eye center and the second preliminary eye center.

One or more embodiments may include none, one, two or more, or all of the following:

The computer system adjusts the capsulotomy pattern to compensate for the tilt-offset of the eye by adjusting a shape of the capsulotomy pattern to compensate for a distortion of the capsulotomy pattern associated with the tilt-offset of the eye.

The computer system adjusts the capsulotomy pattern to compensate for the tilt-offset of the eye by adjusting a z-depth of the capsulotomy pattern to compensate for a distortion of the capsulotomy pattern associated with the tilt-offset of the eye.

The computer system adjusts the capsulotomy pattern to compensate for the tilt-offset of the eye by adjusting the capsulotomy pattern such that a pattern axis of the capsulotomy pattern aligns with an eye axis of the eye.

The ophthalmic system includes a display system that displays the image of the eye. The display system may display an eye axis graphic on the image of the eye indicating an eye axis of the eye. The display system may display an eye center graphic on the image of the eye indicating an eye center of the eye. The display system may display a capsulotomy pattern graphic on the image of the eye indicating a location of the capsulotomy pattern in the image of the eye.

The ophthalmic system includes a display system that displays a graphical user interface that can receive user input. The graphical user interface may display eye axis options and receive the user input comprising a selection of an eye axis option. The graphical user interface may display eye center options and receive the user input comprising a selection of an eye center option.

### BRIEF DESCRIPTION OF THE DRAWINGS

FIG. 1 illustrates an example of an ophthalmic system that may perform a procedure on an eye of a patient, according to at least one embodiment described in the present disclosure;

FIGS. 2A and 2B illustrate another example of an ophthalmic system that may perform a procedure on an eye, according to at least one embodiment described in the present disclosure;

FIG. 3 illustrates an example of a computing system, according to at least one embodiment described in the present disclosure;

FIGS. 4A and 4B illustrate examples of images of an eye that may be displayed by a display device of an ophthalmic system, according to at least one embodiment described in the present disclosure;

FIGS. 5A and 5B illustrate examples of images of an eye and a capsulotomy pattern that may be displayed by a display device of an ophthalmic system, according to at least one embodiment described in the present disclosure;

FIGS. 6A to 6I illustrate diagrams that describe examples of adjusting a capsulotomy pattern to compensate for a tilt of an eye, according to at least one embodiment described in the present disclosure;

FIG. 7 illustrates an example of a graphical user interface (GUI) presented on a display device, according to at least one embodiment described in the present disclosure; and

FIG. 8 illustrates an example of a method to adjust a capsulotomy pattern, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments. For example, various examples of tilt may be exaggerated to better illustrate the changes in shape to illustrate the principles of the various embodiments.

The present disclosure relates to aligning a capsulotomy pattern with an eye to perform a capsulotomy procedure. In one or more embodiments, an ophthalmic system performs a capsulotomy procedure on an eye according to a capsulotomy pattern. The capsulotomy pattern may be, e.g., a circular pattern designed to yield a circle centered on the eye. In some cases, however, the capsulotomy pattern might not be aligned with the eye. For example, the capsulotomy pattern might not be centered on the eye. As another example, the capsulotomy pattern might be tilted relative to the eye. In the one or more embodiments, the ophthalmic system may adjust the capsulotomy pattern to align the pattern with the eye. In these and other embodiments, while referred to as the capsulotomy pattern being misaligned or tilted relative to the eye, it will be appreciated that this may be caused by the eye being slightly tilted during docking, or other misalignment of the eye relative to the laser system during docking.

Certain embodiments of the present disclosure may provide improvements over known ophthalmic systems. For example, the ophthalmic system may adjust the capsulotomy pattern to compensate for an eye that is tilted relative to the ophthalmic system. As another example, the ophthalmic system may adjust the capsulotomy pattern to compensate for an eye that is not centered relative to the ophthalmic system. As yet another example, the ophthalmic system may display the adjusted capsulotomy pattern overlayed onto an image of the eye. As yet another example, the ophthalmic system may allow a user to select an eye axis with respect to which tilt is determined. As yet another example, the ophthalmic system may allow a user to select an eye center with respect to which centering is determined.

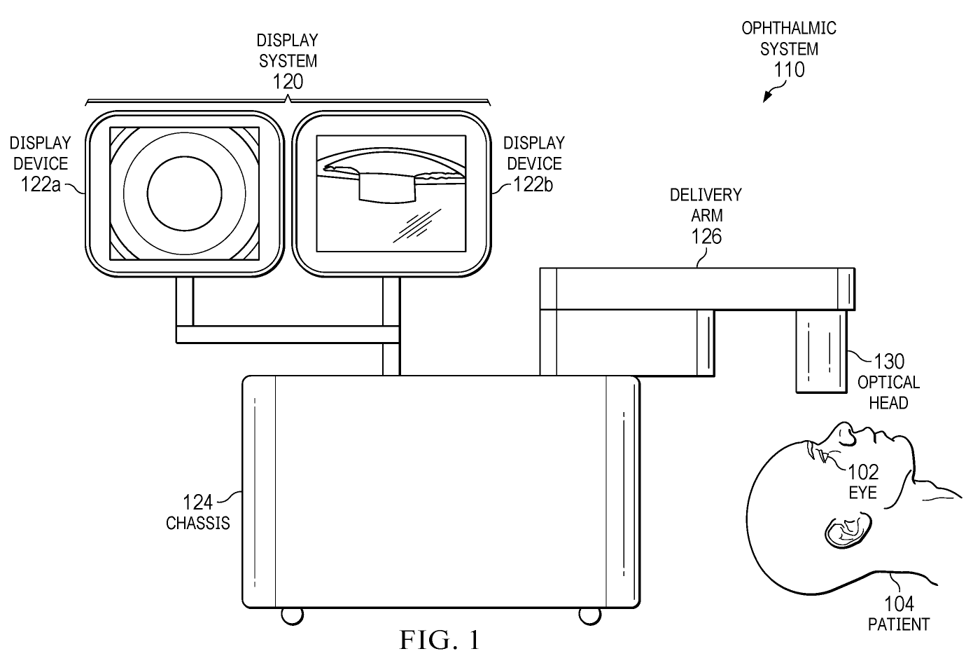

FIG. 1 illustrates an example of an ophthalmic system 110 that may perform a procedure on an eye 102 of a patient 104, according to at least one embodiment described in the present disclosure. In the example, the ophthalmic system 110 includes a display system 120 (with display devices 122 (122a and 122b)), a chassis 124, a delivery arm 126, and/or an optical head 130, which may be coupled (e.g., mechanically, electromagnetically, optically, communicatively, and/or otherwise coupled) as shown.

The ophthalmic system 110 may be any suitable medical device that performs a medical procedure, such as a surgical and/or diagnostic procedure. Examples of medical procedures include refractive, cataract, retinal, or other procedure performed on any suitable part of the eye 112. In one or more embodiments, the ophthalmic system 110 may be a surgical system that directs a laser beam to a part of the eye (e.g., cornea, lens, and/or other eye tissue) to process the tissue (e.g., ablate and/or photodisrupt the tissue). In the example, the ophthalmic system 110 may be a surgical system that performs cataract surgery (e.g., femtosecond laser assisted cataract (FLAC) surgery) to remove a crystalline lens from the eye. In FLAC surgery, a laser (e.g. a femtosecond laser) may be used to create photodisruptions arranged in a fragmentation pattern to segment the crystalline lens in order to facilitate removal of the crystalline lens.

In one or more embodiments, the ophthalmic system 110 may detect the alignment of the eye 102 with the optical head 130 and display the alignment on the display system 120. If the ophthalmic system 110 detects a misalignment, the ophthalmic system 110 may adjust a capsulotomy pattern to compensate for the misalignment, as described in more detail herein.

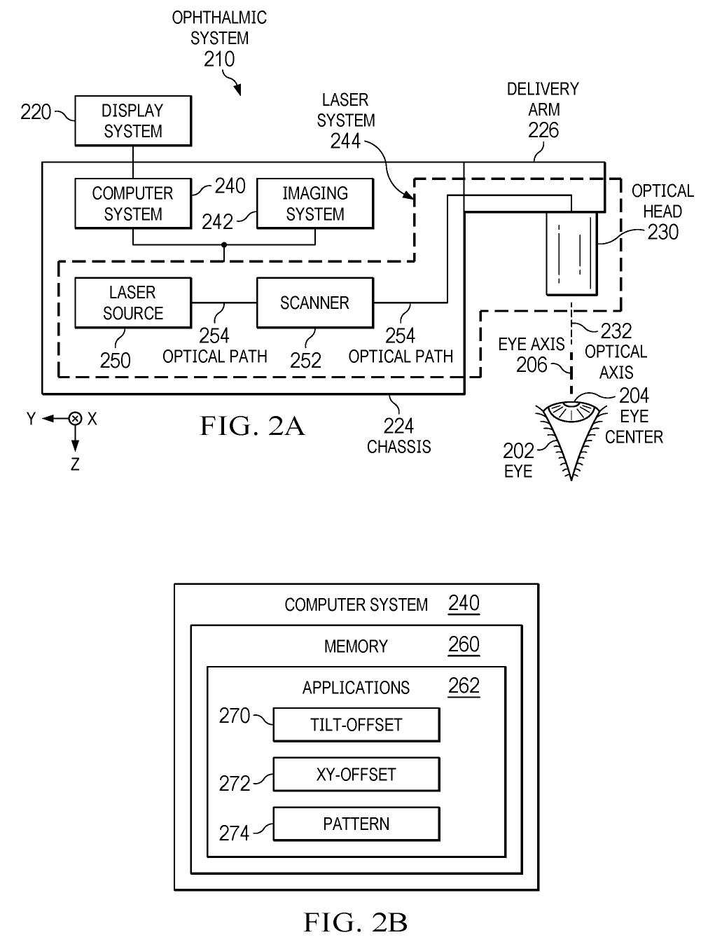

FIGS. 2A and 2B illustrate another example of an ophthalmic system 210 that may perform a capsulotomy procedure on an eye 202, according to at least one embodiment described in the present disclosure. In the example, the eye 204 has an eye center 204 and an eye axis 206. The ophthalmic system 210 includes a display system 220, a chassis 224, a delivery arm 226, an optical head 230 (with an optical axis 232), a computer system 240, an imaging system 242, and/or a laser system 244, which may be coupled as shown. The laser system 244 includes a laser source 250, a scanner 252, an optical path 254, and/or the optical head 230, which may be coupled as shown. The computer system 240 has a memory 260 that stores applications 262, such as a tilt-offset application 270, an xy-offset application 272, and/or a pattern application 274.

For ease of explanation, the embodiments may be described using the following example xyz-coordinate system, which may be regarded as the coordinate system of the ophthalmic system 210, although any suitable coordinate system may be used. In the example, the z-axis is aligned with the optical axis 232 of the laser system 244, and an xy-plane is orthogonal to the z-axis. An example of an xy-plane may be the target plane with which the target (e.g., the eye 202) should be when the target is aligned in the z-direction. Of course, any suitable coordinate system may be used.

In an example overview, the imaging system 242 generates an image of the eye 202. The computer system 240 determines a tilt-offset of the eye from the image, where the tilt-offset of the eye 202 is relative to the z-axis. The computer system 240 adjusts a capsulotomy pattern to compensate for the tilt-offset of the eye and instructs the laser system 244 to provide a laser beam according to the adjusted capsulotomy pattern. In certain embodiments, the computer system 240 may also determine an xy-offset of the eye 202 from an image of the eye 202 and adjust the capsulotomy pattern to compensate for the xy-offset of the eye 202.

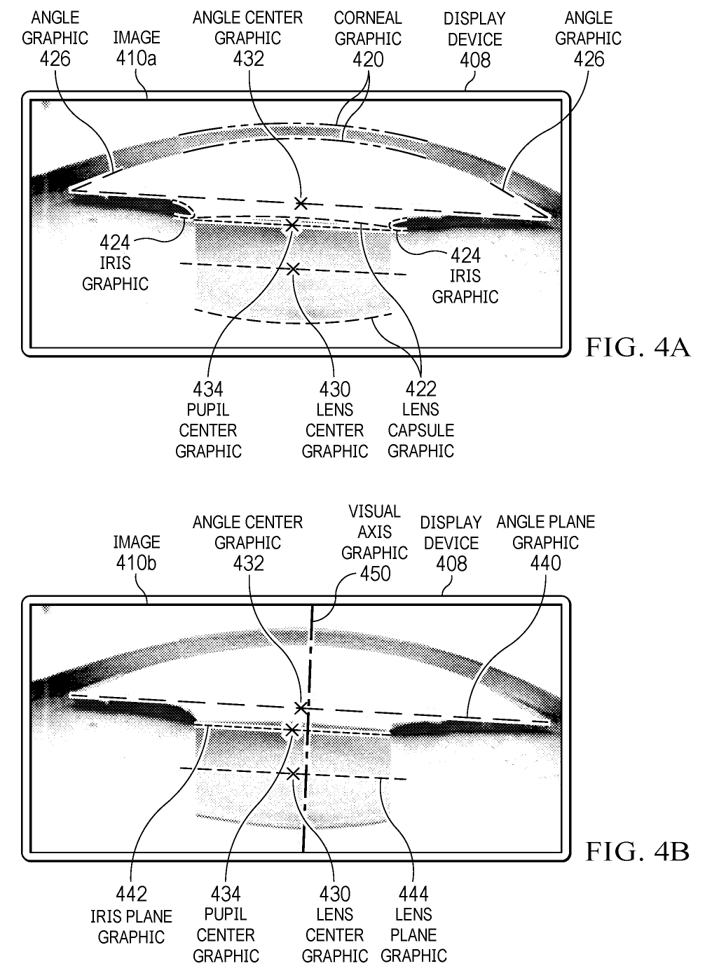

In the example, the eye center 204 may be any suitable center of the eye 202. Examples of an eye center 204 include a capsule center, a pupil center, a limbus center, an angle center, a visual axis center, and/or other center of a structure of the eye, which are described herein (e.g., with reference to FIGS. 4A and 4B). An eye center 204 may also be a function of one or more other eye centers. For example, an eye center 204 may be a predefined distance from an eye center (e.g., the pupil center) or may be a predefined point between multiple eye centers (e.g., a pupil center and a limbus center).

The eye axis 206 may be any suitable axis of the eye 202. Examples of an eye axis 206 include a visual axis, an optical axis, and a pupil center axis, and/or other axis of a structure of the eye, which are described herein (e.g., with reference to FIGS. 4A and 4B). An eye axis 206 may also be a function of one or more other eye axes. For example, an eye axis 206 may be a predefined angle from an eye axis (e.g., the visual axis) or may be a predefined line between multiple eye axes (e.g., a visual axis and an optical axis).

Turning to the components, the chassis 224 houses and/or supports at least a portion of the laser system 244, the imaging system 242, and/or the computer system 240. The laser system 244 directs a laser beam towards the eye 202. The laser system 244 may include a laser source 250 that generates the laser beam and a scanner 252 that guides the laser beam to focus points within the eye 202. The laser source 250 may be a femtosecond laser or other ultrashort pulse laser. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (µm). Examples of ranges include the ultraviolet (e.g., in the range of 180 to 400 nm, such as 190 to 195 nm and/or 345 to 355 nm), visible, or infrared (including near-infrared) wavelength (e.g., in the range of 1000 to 1250 and/or 1250 to 1500 nm).

One or more optical devices direct the laser beam along the optical path 254 towards the eye 202. An optical device can act on (e.g., transmit, reflect, refract, diffract, collimate, condition, shape, focus, modulate, and/or otherwise act on) a laser beam. Examples of optical devices include a lens, prism, mirror, diffractive optical element (DOE), holographic optical element (HOE), and spatial light modulator (SLM).

The delivery arm 226 supports, moves, and/or facilitates movement of the optical head 230. The optical head 230 includes an objective lens that directs the laser beam towards the eye 202. The optical head 230 has an optical axis 232, which may be an optical axis of the objective lens. In one or more embodiments, the optical axis 232 may be used to align the eye 202 with the optical head 230. For example, this may be accomplished by moving the optical head 230 and/or adjusting one or more of the optics along the optical path 254. Additionally or alternatively, this may include adjusting a shot pattern in software such that the shots follow an updated pattern that appears tilted, although still fired from the original position, and may still follow a sequence of layers of shots to compile the adjusted shat pattern.

The imaging system 242 generates an image of the eye 202. In the example, imaging system 242 includes one or more detectors that detect light reflected from the eye 202 to generate an image of the eye 202. A detector of the imaging system 242 may be any suitable detector that detects reflected light. For example, a detector may be a digital camera that records reflections of visible light to generate a real-time image of the eye 202. As another example, a detector may be an optical coherence tomography (OCT) system that can generate a two-dimensional (2D) and/or a three-dimensional (3D) OCT image of the eye 202. In certain examples, a 3D OCT image may include two or more B-scans that can provide information about the tilt of the eye 202 relative to an eye axis 206.

The display system 220 includes one or more display devices. Examples of display devices include computer monitors, display screens, or other devices that can display images of the eye 202 and/or one or more graphical user interfaces (GUIs). In one or more embodiments, the display system 220 may present a real-time image (such as a video) of the eye 202, an OCT image of the eye 202, and/or a GUI.

The computer system 240 send instructions to control the operations of components of the ophthalmic system 210, e.g., the display system 220, the computer system 240, the imaging system 242, and/or the laser system 244. In one or more embodiments, the computer system 240 receives an image of the eye from the imaging system 242 and analyzes the image (using, e.g., image processing software) to determine an xy-offset and/or a tilt-offset of the eye 202. The computer system 240 adjusts the capsulotomy pattern to compensate for the xy-offset and/or the tilt-offset of the eye and instructs the laser system 244 to provide a laser beam according to the adjusted capsulotomy pattern.

The computer system 240 may use the tilt-offset application 270 to determine the tilt-offset in any suitable manner. In one or more embodiments, the computer system 240 may identify an eye plane (e.g., an iris plane or other suitable plane such as described herein such as those described with reference to FIGS. 4A and/or 4B) in the image of the eye. The computer system 240 may then determine the tilt-offset according to the eye plane and the z-axis, e.g., according to an angle of the iris plane relative to the z-axis. In one or more embodiments, the computer system 240 may identify an eye axis (e.g., a visual axis or other suitable axis such as described herein such as those described with reference to FIGS. 4A and/or 4B) from the image of the eye. The computer system 240 may then determine the tilt-offset between the eye axis and the z-axis, e.g., according to an angle of the visual axis relative to the z-axis. Examples of determining the tilt-offset are described herein.

The computer system 240 may use the xy-offset application 272 to determine the xy-offset in any suitable manner. In one or more embodiments, the computer system 240 may identify an eye center (e.g., a pupil center or other suitable eye center such as described herein such as those described with reference to FIGS. 4A and/or 4B) in an image of the eye. The computer system 240 may then determine the xy-offset according to the eye center and a central xy-point, e.g., according to the distance between the pupil center and the central xy-point. The central xy-point may be a point at which a capsulotomy pattern is to be centered, e.g., the point at which the z-axis intersects the target plane.

The computer system 240 may use the pattern application 274 to adjust a capsulotomy pattern to compensate for an xy-offset and/or tilt-offset. The capsulotomy pattern may be adjusted in any suitable manner. For example, the capsulotomy pattern may be adjusted such that a pattern center aligns with an eye center. As another example, the capsulotomy pattern may be adjusted such that a pattern axis aligns with the eye axis. In the example, the shape of the capsulotomy pattern may be adjusted to compensate for a distortion of the capsulotomy pattern associated with the tilt-offset. This may involve adjusting a z-depth of the capsulotomy pattern as described herein.

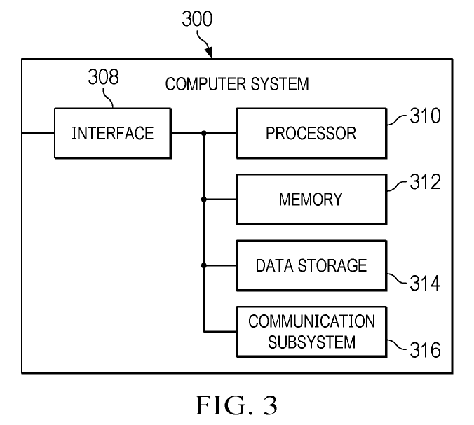

FIG. 3 illustrates an example of a computing system 300, according to at least one embodiment described in the present disclosure. The computing system 300 may include an interface 308, a processor 310, a memory 312, a data storage 314, and/or a communication subsystem 316, any or all of which may be communicatively coupled. Any or all of the computing system 300 may be implemented as computer hardware and/or software. Any or all of the computer systems described herein may be implemented as a computing system consistent with the computing system 300.

In the example, the interface 308 may receive input to the computing system 300 and/or send output from the computing system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computing entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the computing system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the computing system 300 without departing from the scope of the present disclosure. For example, the computing system 300 may include more or fewer components than those explicitly illustrated and described.

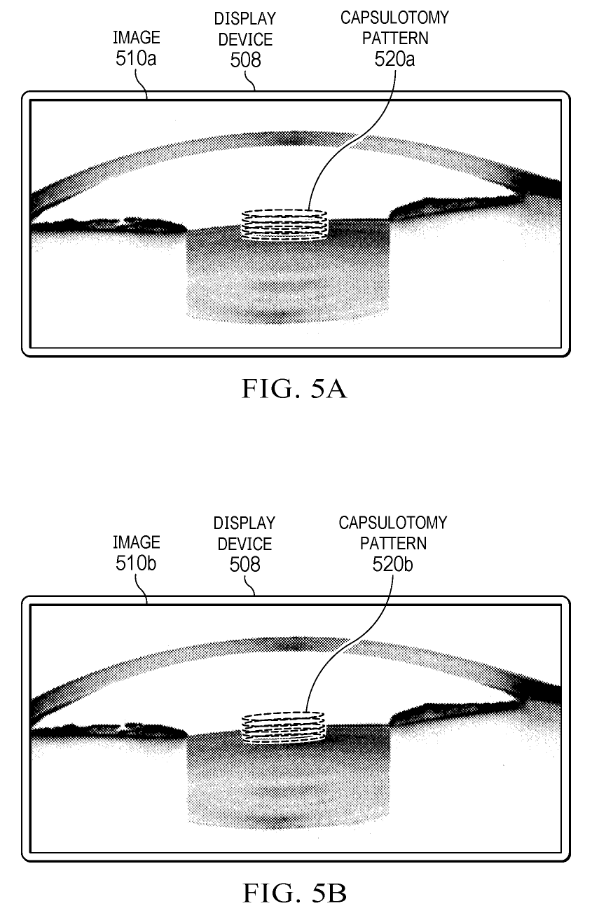

FIGS. 4A to 5B illustrate examples of images (410a to 510b, respectively) of an eye that may be displayed by a display device of an ophthalmic system, according to at least one embodiment described in the present disclosure. An image may be generated in any suitable manner with any suitable light. For example, an image may be a visible light image, an infrared image, and/or an OCT image. An image may be a 2D image and/or a 3D image. An image may include one or more frames of the image of the object. For example, an image may be a still image that includes one frame of the image. As another example, an image may be a moving image (such as a video) that includes a sequence of frames of the image. An image may include any suitable graphic overlaid onto the image. Examples of graphics include an eye axis graphic indicating an eye axis of the eye, an eye center graphic indicating an eye center of the eye, and/or a capsulotomy pattern graphic indicating a location of the capsulotomy pattern in the eye.

FIGS. 4A and 4B illustrate examples of images 410 (410a and 410b, respectively) of an eye that may be presented on a display device 408 of an ophthalmic system, according to at least one embodiment described in the present disclosure. The images 410 (410a and 410b) include an OCT image of the eye with graphics indicating features of the eye.

Image 410a includes eye structure graphics that indicate locations of eye structures of the eye (e.g., a cornea, a lens, and/or an iris). In the example, a cornea graphic 420 indicates an anterior surface and a posterior surface of a cornea. A lens capsule graphic 422 indicates an anterior surface and a posterior surface of a lens capsule. An iris graphic 424 indicates an interior circumference of an iris. An angle graphic 426 indicates a corneal angle between a posterior surface of the cornea and an anterior surface of the iris. Image 410a also includes eye center graphics that indicate eye centers of the eye. For example, a lens center graphic 430 indicates a lens center, an angle center graphic 432 indicates an angle center, and a pupil center graphic 434 indicates a pupil center. In these and other embodiments, the various graphics may include overlays or other markings on the image 410. For example, the lens center graphic 430, the angle center graphic 432, and/or the pupil center graphic 434 may include an “X” overlayed atop the image 410. As another example, the lens capsule graphic 422 may include one or more lines of various weights, dashes, etc. which may be overlaid on the image 410.

The computer system may determine an eye center from the eye structures of the image 410a in any suitable manner. For example, a pupil may be identified according to an iris (indicated by the iris graphic 424). The computer system may determine a pupil center (indicated by the pupil center graphic 434) as a center point of the pupil. As another example, a lens capsule (indicated by the lens capsule graphic 422) may be identified. The computer system may then determine a lens capsule center (indicated by the lens center graphic 430) as a center point of the lens capsule. As another example, corneal angles (indicated by the angle graphic 426) may be identified. The computer system may then determine an angle center (indicated by the angle center graphic 432) as a center point between the vertices of the corneal angles. As yet another example, an eye axis may be identified (e.g., as described with reference to FIG. 4B). The computer system may determine an eye center according to the eye axis. In one or more cases, an eye center may be where the eye axis intersects an eye plane (e.g., as described with reference to FIG. 4B).

In one or more embodiments, a computer system may identify an eye center according to a function of one or more eye centers, as described herein. For example, a computer system may identify a first preliminary eye center in the image of the eye according to one approach (e.g., the pupil center), identify a second preliminary eye center in the image according to another approach (e.g., the angle center), and determine the eye center according to a function (e.g., an average or weighted average function) of the first preliminary eye center and the second preliminary eye center.

Image 410b includes eye plane graphics indicating various example eye planes of the eye. In the example, an angle plane graphic 440 indicates an angle plane, an iris plane graphic 442 indicates an iris plane, and the lens plane graphic 444 indicates a lens plane. An eye plane may be a planar feature of a structure of the eye or a function of one or more other eye planes. For example, an eye plane may be an average plane between the lens plane and the angle plane.

A computer system may determine an eye plane of the eye in any suitable manner. For example, an iris plane (indicated by an iris plane graphic 442) may be a plane defined by the iris (indicated by the iris graphic 424). As another example, a lens plane (indicated by the lens plane graphic 444) may be a plane that substantially bisects the lens (indicated by the lens capsule graphic 422) such that the plane is substantially parallel to the pupil. As another example, an angle plane (indicated by an angle plane graphic 440) may be a plane defined by the vertices of the corneal angles (indicated by the angle graphic 426).

Image 410b also includes an eye axis graphic indicating an eye axis of the eye. In the example, the visual axis graphic 450 indicates the visual axis as the eye axis. In one or more embodiments, the computer system may determine an eye axis from structures of the eye in any suitable manner. A visual axis may be determined in any suitable manner. For example, a visual axis may be a line through a corneal vertex and a nodal point. The corneal vertex may be the highest point of the cornea, and the nodal point may be the center of the corneal curvature, both of which may be determined from the cornea graphic 420. As another example, a visual axis may be a line through the nodal point and the fovea, which may be identified in a back-of-the-eye image of the eye (not shown).

An optical axis may be determined in any suitable manner as another example of an eye axis. For example, an optical axis may be determined as a line orthogonal to a cornea surface (indicated by the cornea graphic 420) that intersects the pupil center, which may be identified as described herein. As another example, an optical axis may be determined as a line normal to at least two surfaces of the eye, e.g., the anterior and/or posterior surfaces of the cornea and/or the lens, which may be identified according to the cornea graphic 420 and/or the lens capsule graphic 422, respectively. A pupillary axis may be determined in any suitable manner as another example of an eye axis. For example, a pupillary axis may be determined as a line that connects the pupil center and the nodal point, which may be identified as described herein.

In one or more embodiments, a computer system may identify the eye axis according to a function of one or more eye axes, as described herein. For example, a computer system may identify a first preliminary eye axis using the image of the eye according to one approach (e.g., the visual axis), identify a second preliminary eye axis using the image according to another approach (e.g., the optical axis), and determine the eye axis according to a function (e.g., an average or weighted average function) of the first preliminary eye axis and the second preliminary eye axis.

FIGS. 5A and 5B illustrate examples of images 510 (510a and 510b, respectively) of an eye and a capsulotomy pattern 520 (520a and 520b, respectively) that may be presented on a display 508 of an ophthalmic system, according to at least one embodiment described in the present disclosure. In the examples, the general shape of the capsulotomy pattern 520 (520a and 520b) is illustrated using dashed lines. That is, the dashed lines are intended to show the general shape of the capsulotomy pattern 520 (520a and 520b), not, e.g., the locations of the laser spots resulting from the capsulotomy pattern 520 (520a and 520b). In one or more embodiments, a laser system may use the capsulotomy pattern to direct a laser beam to perform a capsulotomy procedure. The capsulotomy pattern starts with a scanned circle located below the anterior capsule. The next circle may be scanned a 1 to 5 microns above the first circle. As each circle is completed, a cylindrical incision is created.

Image 510a illustrates a capsulotomy pattern 520a, and image 510b illustrates an adjusted capsulotomy pattern 520b. The capsulotomy pattern 520a may be adjusted to compensate for an xy-offset and/or tilt-offset to yield the adjusted capsulotomy pattern 520b. The capsulotomy pattern 520a may be adjusted in any suitable manner. For example, the capsulotomy pattern 520a may be adjusted such that a pattern center aligns with an eye center. As another example, the capsulotomy pattern 520a may be adjusted such that a pattern axis aligns with the eye axis. Examples of adjusting the capsulotomy pattern 520a are described herein.

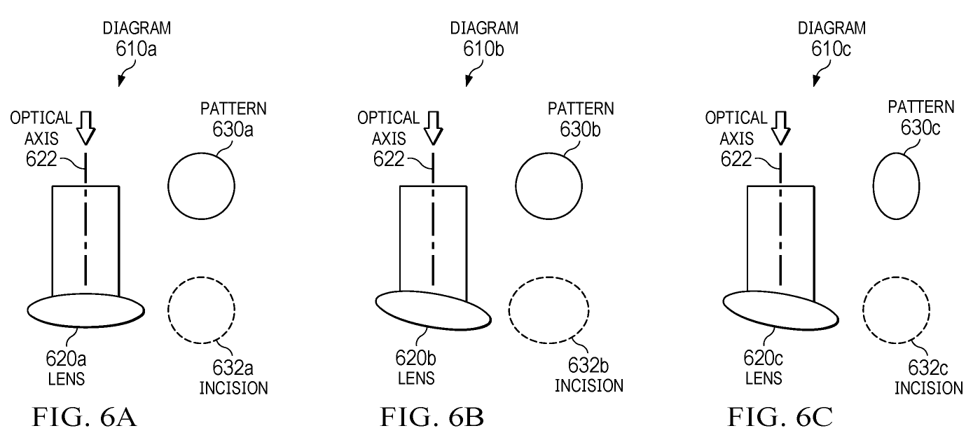

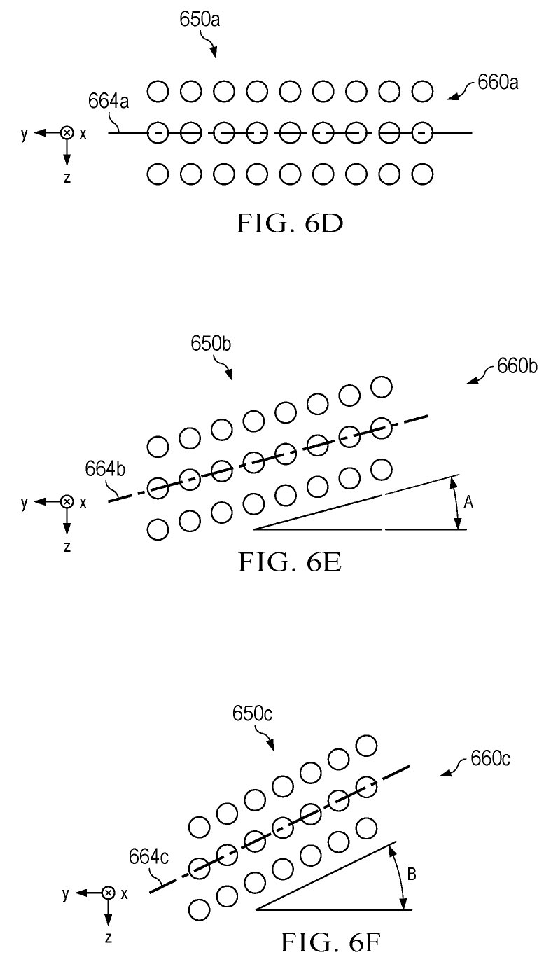

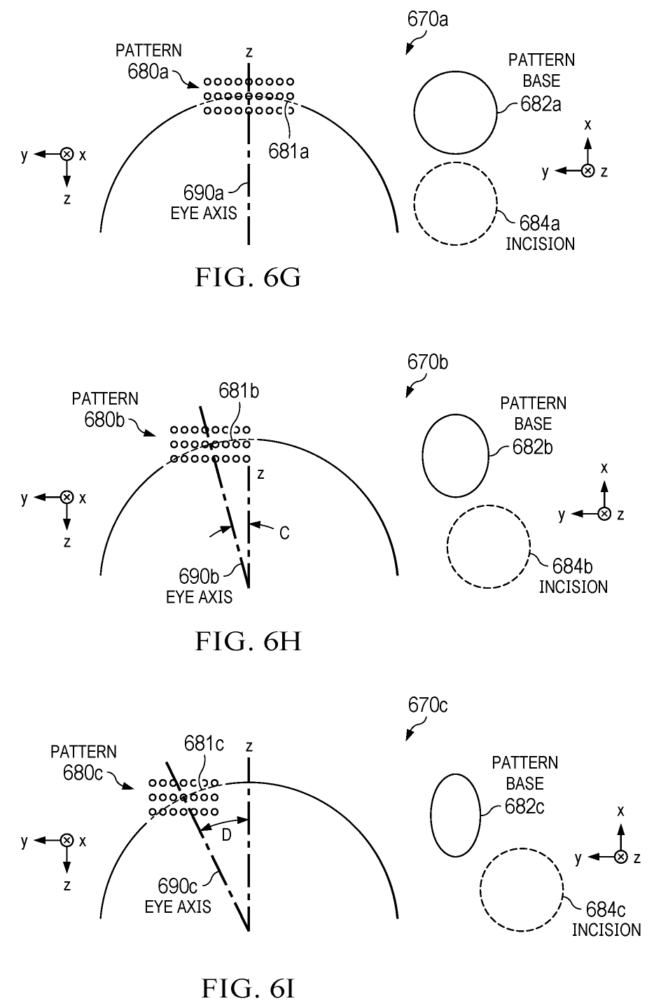

FIGS. 6A to 6I illustrate examples of diagrams 610 (610a to 610c, 650a to 650c, and 670a to 670c, respectively) that describe adjusting a capsulotomy pattern to compensate for a tilt of an eye, according to at least one embodiment described in the present disclosure. The capsulotomy pattern may have any suitable shape, size, and/or location. In at least one embodiment, the capsulotomy pattern may be shaped like an elliptical cylinder (e.g., a circular cylinder) that has an axis, elliptical (e.g., circular) bases, and a midplane that bisects the axis and is parallel to the bases. A base of the cylinder may be sized relative to the optical zone of, e.g., an IOL to be inserted into the eye, such as smaller than or the same size as the IOL optical zone. The capsulotomy pattern may be centered relative to an eye center and/or an eye axis.

In the examples, the general shape of the capsulotomy pattern is illustrated using dots. That is, the dots are intended to show the general shape of the capsulotomy pattern, not, e.g., the locations of the laser spots resulting from the capsulotomy pattern. For example, the resulting incision may have more laser spots, the same number of laser spots, or fewer laser spots than the number of dots shown in the illustrations. In these and other embodiments, the dots may be used to provide an example of the way in which the laser shat patterns may be adjusted at the level of detail of the laser spots, although the adjustment need not be in the manner illustrated.

In one or more embodiments, a computer system of an ophthalmic system described herein (e.g., as described with reference to FIG. 2A) may perform the adjustment of the capsulotomy pattern. The computer system may adjust the capsulotomy pattern to compensate for a tilt of an eye relative to an xyz-coordinate system of the ophthalmic system. The capsulotomy pattern may be adjusted in any suitable manner, e.g., a shape and/or a tilt of the pattern may be adjusted.

FIGS. 6A to 6C include diagrams 610 (610a to 610c, respectively) that illustrate examples of how a capsulotomy pattern may be adjusted to compensate for a tilt of an eye. The diagrams 610 (610a to 610c) show a lens 620 (620a to 620c, respectively) positioned relative to an optical axis 622. The diagrams 610 (610a to 610c) also show a capsulotomy pattern 630 (630a to 630c, respectively) and a corresponding incision 632 (632a to 632c, respectively) resulting from the capsulotomy pattern 630 (630a to 630c, respectively).

The diagram 610a shows a lens 620a that is properly aligned with the optical axis 622. The circular capsulotomy pattern 630a yields the expected circular incision 632a.

The diagram 610b shows a lens 620b that is tilted relative to the optical axis 622. Due to the tilt, the circular capsulotomy pattern 630b yields an elliptical incision 632b.

The diagram 610c shows a lens 620c that is tilted relative to the optical axis 622. In this case, the shape of the capsulotomy pattern 630c has been adjusted to an ellipse to yield the expected circular incision 632c. This may also involve adjusting a z-depth of the capsulotomy pattern to compensate for a distortion of the capsulotomy pattern associated with the tilt-offset.

FIGS. 6D to 6F include diagrams 650 (650a to 650c, respectively) that illustrate examples of adjusting a capsulotomy pattern, e.g., adjusting a tilt of the pattern, to compensate for a tilt of an eye. In the examples, the capsulotomy pattern 660 (660a to 660c) is shaped as a circular cylinder with a midplane 664 (664a to 664c, respectively). In the examples, the tilt of an eye may be described as an angle X of an eye plane of the eye relative to an xy-plane of the xyz-coordinate system of the ophthalmic system.

The diagram 650a shows a capsulotomy pattern 660a for an eye that is not tilted, e.g., the eye plane of the eye is substantially parallel to the xy-plane of the xyz-coordinate system. Since the eye is not tilted, the capsulotomy pattern 660a does not need to be adjusted to compensate for tilt. The midplane 664a is the substantially parallel to the xy-plane and the eye plane.

The diagram 650b shows a capsulotomy pattern 660b for an eye that is tilted by an angle A, where the angle A may be any suitable angle (e.g., an angle in a range of 1 to 20 degrees). The capsulotomy pattern 660b is adjusted to compensate for the tilt. The midplane 664b is adjusted to the angle A (as shown in the capsulotomy pattern 660b) such that the midplane 664b is substantially parallel to the eye plane.

The diagram 650c shows a capsulotomy pattern 660c for an eye that is tilted by an angle B, where the angle B may be any suitable angle (e.g., an angle in a range of 10 to 40 degrees). The capsulotomy pattern 660c is adjusted to compensate for the tilt. The midplane 664c is adjusted to the angle B (as shown in the capsulotomy pattern 660c) such that the midplane 664c is substantially parallel to the eye plane.

In some embodiments the adjustment may include lifting or lowering each successive column of laser spots such that a middle of a row of spots follows the midplane 664.

FIGS. 6G to 6I include diagrams 670 (670a to 670c, respectively) that illustrate examples of adjusting a capsulotomy pattern, e.g., adjusting a shape of the pattern, to compensate for a tilt of an eye. In the examples, the capsulotomy pattern is shaped as a circular cylinder with a circular base designed to yield a circular incision. However, as described with reference to FIG. 6B, the tilt of the eye may yield an unintended elliptical incision if the circular base is used. The diagrams 670b and 670c show how the shape of the circular base may be adjusted to compensate for the tilt of an eye.

The diagrams 670 (670a to 670c) show the capsulotomy pattern 680 (680a to 680c, respectively) as a side view, a pattern base 682 (682a to 682c, respectively) of the capsulotomy pattern, and an incision 684 (684a to 684c, respectively) resulting from the capsulotomy pattern. The side view also shows an anterior surface 681 (681a to 681c, respectively) of the cornea. In the examples, the tilt of an eye may be described as an angle X of an eye axis 690 (690a to 690c) of the eye relative to a z-axis of an xyz-coordinate system of an ophthalmic system.

The diagram 670a shows a capsulotomy pattern 680a for an eye that is not tilted, e.g., the eye axis 690a is substantially parallel to the z-axis of the xyz-coordinate system. The circular pattern base 682a yields a circular incision 684a, as designed. The capsulotomy pattern 680a is not adjusted.

The diagram 670b shows a capsulotomy pattern 680b for an eye that is tilted by an angle C, where the angle C may be any suitable angle (e.g., an angle in a range of 1 to 20 degrees). The shape of the pattern base 682b is adjusted to an elliptical shape to compensate for the tilt, which yields a circular incision 684b. In some embodiments, one or more columns of laser shots may be removed from the pattern 680b to maintain a relative size of the incision 684b. For example, the pattern 680b includes fewer columns of dots than the pattern 680a as illustrated.

The diagram 670c shows a capsulotomy pattern 680c for an eye that is tilted by an angle D, where the angle D may be any suitable angle (e.g., an angle in a range of 10 to 40 degrees). The shape of the pattern base 682c is adjusted to an elliptical shape to compensate for the tilt, which yields a circular incision 684c. If the angle D is greater than the angle C, the difference between the major axis and the minor axis of the pattern base 682c (for the angle D) is greater than the difference between the major axis and the minor axis of the pattern base 682b (for the angle C). In other words, the elliptical shape of the pattern base 682c for the larger angle is “thinner” than the elliptical shape of the pattern base 682c for the smaller angle. Stated another way, in some embodiments, one or more columns of laser shots may be removed from the pattern 680c to maintain a relative size of the incision 684c. For example, the pattern 680c includes fewer columns of dots than the pattern 680b as illustrated.

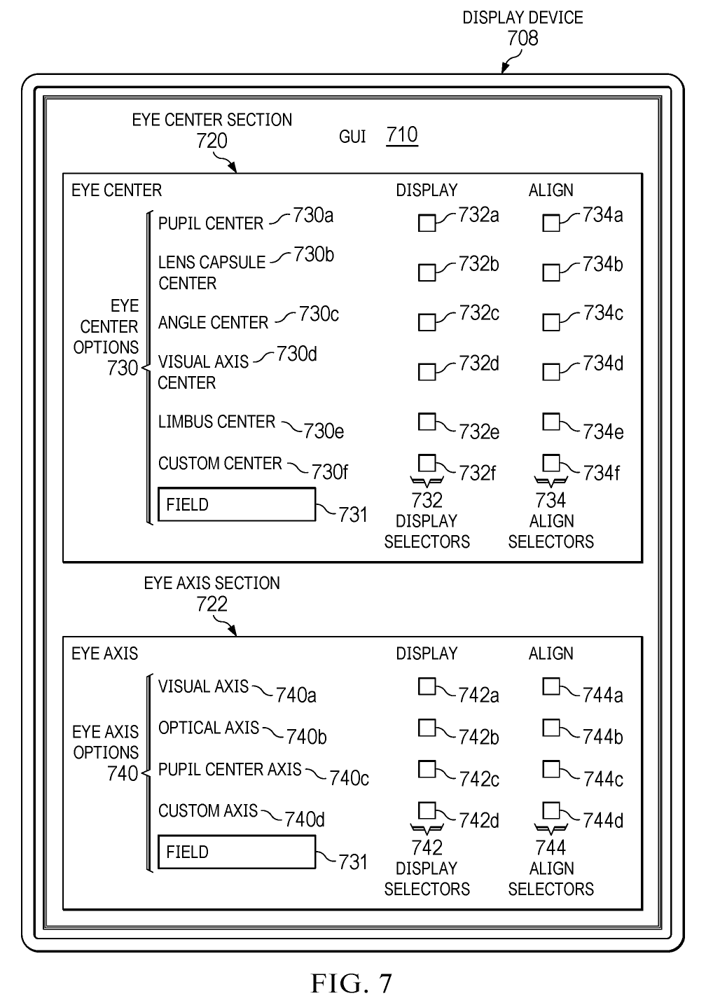

FIG. 7 illustrates an example of a graphical user interface (GUI) 710 presented on a display device 708, according to at least one embodiment described in the present disclosure. The GUI 710 includes an eye center section 720 and an eye axis section 722. The eye center section 720 displays eye center options 730 (730a to 730f) and can receive a selection of an eye center option 730 (730a to 730f). The eye center options 730 include a pupil center 730a, a lens capsule center 730b, an angle center 730c, a visual axis center 730d, a limbus center 730e, and/or a custom center 730f. The custom center 730f may have a field 731 where a user may enter a customized eye center. For example, an eye center may be a function of one or more other eye centers, as described herein.

The eye center section 720 includes display selectors 732 (732a to 732f) and/or align selectors 734 (734a to 734f). In the example, each eye center option 730 (730a to 730f) has a corresponding display selector 732 (732a to 732f, respectively) and/or a corresponding align selector 734 (734a to 734f, respectively). A user may select a display selector 732 (732a to 732f) to display the eye center corresponding to the selected eye center option 730 (730a to 730f, respectively) on an image of the eye. A user may select an align selector 734 (734a to 734f) to use the eye center corresponding to the selected eye center option 730 (730a to 730f, respectively) to align a capsulotomy pattern in an xy-plane.

The eye axis section 722 displays eye axis options 740 (740a to 740d) and receives a selection of an eye axis option 740 (740a to 740d). The eye axis options 740 (740a to 740d) include a visual axis 740a, an optical axis 740b, a pupil center axis 740c, and/or a custom axis 740d. The custom axis 740d may have a field 741 where a user may enter a customized eye axis. For example, an eye axis may be a function of one or more other eye axis, as described herein.

The eye axis section 722 also includes display selectors 742 (742a to 742f) and/or align selectors 744 (744a to 744f). In the example, each eye axis option 740 (740a to 740f) has a corresponding display selector 742 (742a to 742d, respectively) and/or a corresponding align selector 744 (744a to 744d, respectively). A user may select a display selector 742 (742a to 742d) to display the eye axis corresponding to the selected eye axis option 740 (740a to 740f, respectively) on an image of the eye. A user may select an align selector 744 (744a to 744f) to use the eye axis corresponding to the selected eye axis option 740 (740a to 740f, respectively) to align a capsulotomy pattern.

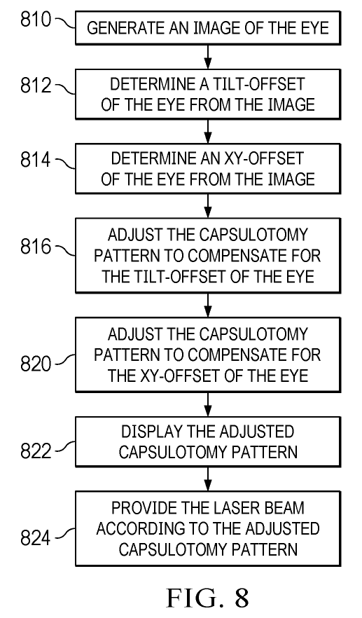

FIG. 8 illustrates an example of a method to adjust a capsulotomy pattern, according to at least one embodiment described in the present disclosure. The method may be performed by any suitable system, such as an ophthalmic system described herein. In the example, the method is performed by an ophthalmic system that includes an imaging system, a computer system, and/or a laser system. The laser system may have an optical axis that may be used to define a z-axis and a central xy-point.

At block 810, the imaging system generates an image of the eye. For example, the imaging system may generate an OCT image of the eye.

At block 812, the computer system determines a tilt-offset of the eye from the image. The computer system may determine the tilt-offset in any suitable manner. As an example, the computer system may identify an eye plane (e.g., an iris plane) in the image of the eye and determine the tilt-offset according to the eye plane relative to the z-axis of the laser system. As another example, the computer system may identify an eye axis (e.g., a visual axis) in the image of the eye and determine the tilt-offset according to the eye axis relative to the z-axis of the laser system.

At block 814, the computer system determines an xy-offset of the eye. The computer system may determine the xy-offset in any suitable manner. For example, the computer system may identify an eye center in an image of the eye and determine the xy-offset according to the eye center relative to the central xy-point. The image may be the OCT image that was used at block 812 to determine the tilt-offset of the eye or other suitable image of the eye, such as a microscope image of the eye.

At block 816, the computer system adjusts the capsulotomy pattern to compensate for the tilt-offset of the eye. For example, the capsulotomy pattern may be adjusted such that an axis of the capsulotomy pattern aligns with the eye axis. In addition, the shape of the capsulotomy pattern may be adjusted to compensate for a distortion of the capsulotomy pattern associated with the tilt-offset.

At block 820, the computer system adjusts the capsulotomy pattern to compensate for the xy-offset of the eye. For example, the capsulotomy pattern may be adjusted such that a center of the capsulotomy pattern aligns with an eye center.

At block 822, the display system displays the adjusted capsulotomy pattern. The display may show the adjusted capsulotomy pattern overlayed onto a real-time image of the eye.

At block 824, the laser system provides the laser beam according to the adjusted capsulotomy pattern to perform a capsulotomy procedure.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. An ophthalmic system comprising:
a laser system configured to provide a laser beam to perform a capsulotomy procedure on an eye, the laser system having an optical axis;
an imaging system configured to generate an image of the eye; and
a computer system configured to:
determine a tilt-offset of the eye from the image of the eye, the tilt-offset of the eye relative to a z-axis corresponding to the optical axis;
adjust a capsulotomy pattern to compensate for the tilt-offset of the eye; and
instruct the laser system to provide the laser beam according to the capsulotomy pattern adjusted to compensate for the tilt-offset of the eye.

2. The ophthalmic system of claim 1, the computer system configured to determine the tilt-offset of the eye from the image of the eye by:
identifying an eye structure in the image of the eye; and
determining the tilt-offset of the eye according to the eye structure and the z-axis.

3. The ophthalmic system of claim 2, the eye structure comprising a lens of the eye.

4. The ophthalmic system of claim 2, the eye structure comprising an iris of the eye.

5. The ophthalmic system of claim 2, the eye structure comprising a cornea of the eye.

6. The ophthalmic system of claim 1, the computer system configured to determine the tilt-offset of the eye from the image of the eye by:
identifying an eye axis from the image of the eye; and
determining the tilt-offset of the eye according to the eye axis and the z-axis.

7. The ophthalmic system of claim 6, the computer system configured to identify the eye axis from the image of the eye by:
identifying a vertex of a cornea in the image of the eye;
identifying a nodal point of the cornea in the image of the eye; and
determining a visual axis according to the vertex of the cornea and the nodal point of the cornea as the eye axis.

8. The ophthalmic system of claim 6, the computer system configured to identify the eye axis from the image of the eye by:
identifying a fovea of a retina in the image of the eye;
identifying a nodal point of a cornea in the image of the eye; and
determining a visual axis according to the fovea of the retina and the nodal point of the cornea as the eye axis.

9. The ophthalmic system of claim 6, the computer system configured to identify the eye axis from the image of the eye by:
identifying a corneal surface of a cornea in the image of the eye;
identifying a lenticular surface of a lens in the image of the eye; and
determining an optical axis according to the corneal surface of the cornea and the lenticular surface of the lens as the eye axis.

10. The ophthalmic system of claim 6, the computer system configured to identify the eye axis from the image of the eye by:
identifying a pupil center of a pupil in the image of the eye;
identifying a nodal point of a cornea in the image of the eye; and
determining a pupillary axis according to the pupil center of the pupil and the nodal point of the cornea as the eye axis.

11. The ophthalmic system of claim 6, the computer system configured to identify the eye axis from the image of the eye by:
identifying a first preliminary eye axis in the image of the eye;
identifying a second preliminary eye axis in the image of the eye; and
determining the eye axis according to the first preliminary eye axis and the second preliminary eye axis.

12. The ophthalmic system of claim 6, the computer system configured to identify the eye axis from the image of the eye by:
receiving a selection of the eye axis; and
using the selection as the eye axis to identify in the image of the eye.

13. The ophthalmic system of claim 1, the computer system configured to:
determine an xy-offset of the eye from the image of the eye, the xy-offset of the eye relative to a central xy-point of an xy-plane defined relative to the z-axis; and
adjust the capsulotomy pattern to compensate for the xy-offset of the eye.

14. The ophthalmic system of claim 13, the computer system configured to determine the xy-offset of the eye from the image of the eye by:
identifying an eye center from the image of the eye; and
determining a difference between the eye center and the central xy-point.

15. The ophthalmic system of claim 1, the computer system configured to:
identify an eye center from the image of the eye; and
determine an xy-offset of the eye according to the eye center and a central xy-point of an xy-plane defined relative to the z-axis.

16. The ophthalmic system of claim 15, the computer system configured to identify the eye center from the image of the eye by:
identifying a pupil in the image of the eye; and
determining a pupil center of the pupil as the eye center.

17. The ophthalmic system of claim 15, the computer system configured to identify the eye center from the image of the eye by:
identifying a lens capsule in the image of the eye; and
determining a lens capsule center of the lens capsule as the eye center.

18. The ophthalmic system of claim 15, the computer system configured to identify the eye center from the image of the eye by:
identifying a plurality of corneal angles in the image of the eye, a corneal angle of the plurality of corneal angles formed from a posterior surface of a cornea of the eye and an anterior surface of an iris of the eye; and
determining an angle center according to the plurality of corneal angles as the eye center.

19. The ophthalmic system of claim 15, the computer system configured to identify the eye center from the image of the eye by:
determining an eye axis of the image of the eye; and
determining the eye center according to the eye axis.

20. The ophthalmic system of claim 15, the computer system configured to identify the eye center from the image of the eye by:
receiving a selection of the eye center; and
identifying the selection as the eye center to identify in the image of the eye.
